(19) United States
(12) Patent Application Publication
(10) Pub. No.: US 2024/0207467 A1
(43) Pub. Date: Jun. 27, 2024

(54) Sanitizing Radiation Apparatus

(71) Applicant: John Marshall, Beverly Hills, CA (US)

(72) Inventor: John Marshall, Beverly Hills, CA (US)

(21) Appl. No.: 18/087,591

(22) Filed: Dec. 22, 2022

(51) Int. Cl. A61L 2/10; A61L 2/24

(52) U.S. Cl. CPC A61L 2/10; A61L 2/24

(57) ABSTRACT

A surface-contaminated sanitizing apparatus is configured to emit energy, particularly user-safe 222 nanometer UV light, for sanitizing a surface, such as, a door handle, a bank of elevator floor selectors, and other potentially unsanitary surfaces that human beings touch repeatedly. This apparatus may be configured to operate automatically in response to detection of a user's finger or hand contacting/touching/nearing such a surface, and to sanitize the surface after any such contact.

A surface-contaminated sanitizing apparatus is configured to emit energy, particularly user-safe 222 nanometer UV light, for sanitizing a surface, such as, a door handle, a bank of elevator floor selectors, and other potentially unsanitary surfaces that human beings touch repeatedly. This apparatus may be configured to operate automatically in response to detection of a user's finger or hand contacting/touching/nearing such a surface, and to sanitize the surface after any such contact.

### SUMMARY OF THE INVENTION

A sanitizing apparatus is configured, upon activation, to emit UV light at a wavelength in the range of about 200 to about 225 nanometers, preferably about 222 nanometers, from a suitable UV light source in the apparatus, and to direct such UV light onto a surface that repeatedly/regularly/often comes into contact with human hands/fingers, and is, for that reason, in part, at least potentially unsanitary. The emitted UV light at, say, 222 nanometers, is trained on substantially the entirety of the affected surface for a time sufficient to sanitize the surface. After that time, the light emission ceases, and the apparatus may reset for another activation. Alternatively, or in addition, the apparatus may be turned on and off by activation of a switch for the apparatus by a user. Compare the system disclosed in U.S. Pat. No. 10,069,992, which uses exclusively a UV source of 253 nanometers or more. Such sources may cause harm if used improprerly.

In some embodiments, the apparatus may include a plurality of UV light sources, including at least one UV source that emits UV light at a wavelength of 253 nanometers, or higher. The apparatus may then be configured to emit UV light at a single, desired wavelength, such as 253 nanometers, or to emit UV light at two or more different wavelengths, such as, 222 nanometers, and 253 nanometers, concurrently, or seriatim, where robust sanitizing is necessary or desirable. The apparatus may, in these circumstances, include a selector switch for activating operation for these purposes.

Among the surfaces that may be sanitized by this apparatus are: banks of elevator floor selectors, door handles, trash chute handles, ATM machines, POS machines, gas pumps among others.

The apparatus may be activated automatically upon detection, within the field of the apparatus's light emission, of the presence of a human being's hand, fingers, or other body part, or by touching the surface to be sanitized, and may then remain activated for a fixed/adjustable period of time sufficient to obtain more than 90% sanitizing of the surface.

### DESCRIPTION OF THE DRAWINGS

Exemplary embodiments of the invention appear in the drawings, and are described in the following detailed description of the drawings, in which:

FIG. 1 is a perspective view of a door that includes an exemplary embodiment plan view of the sanitizing apparatus of this invention;

FIG. 2 is a front view of the door depicted in FIG. 1;

FIG. 3 is a side view of the door depicted in FIG. 1;

FIG. 4 is a partial, enlarged perspective view of the door depicted in FIG. 1, illustrating a lower sanitizing assembly;

FIG. 5 is a partial, enlarged perspective view of the door depicted in FIG. 1, illustrating an upper sanitizing assembly;

FIG. 6 is an exploded perspective view of a housing assembly; and

FIG. 7 is a schematic view of the electrical components of the sanitizing apparatus depicted in FIGS. 1 to 6.

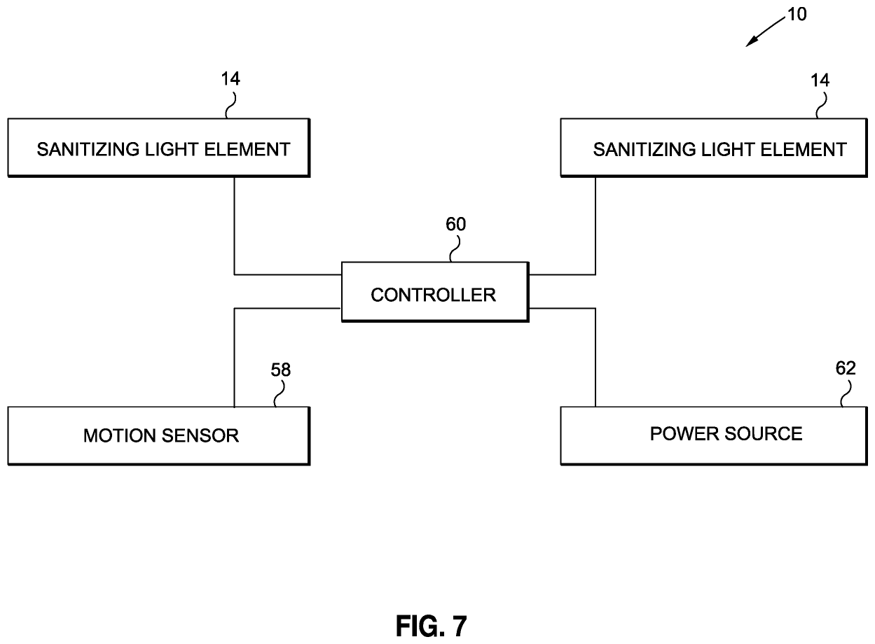

### DETAILED DESCRIPTION OF THE DRAWINGS

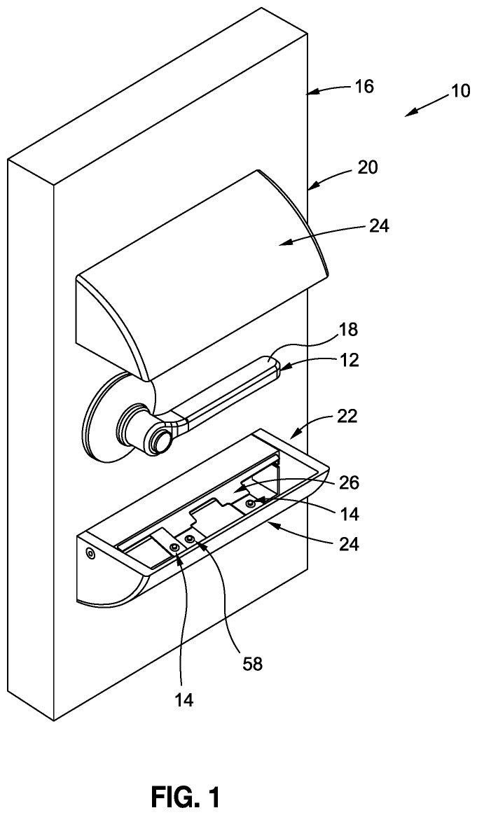

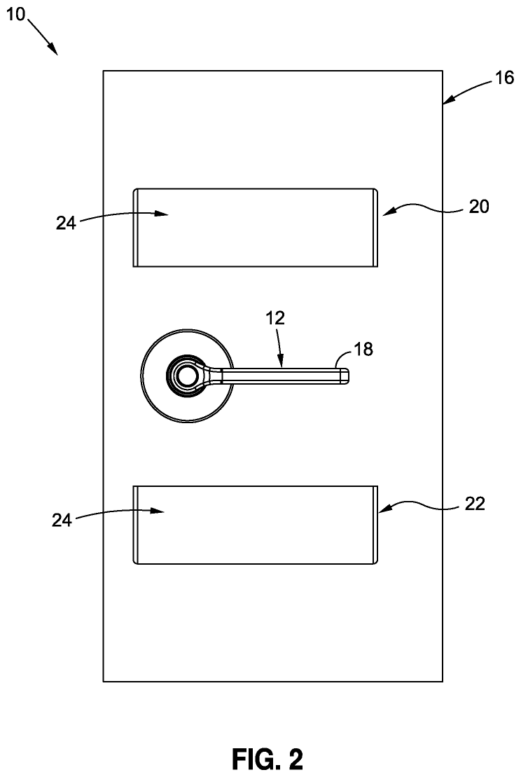

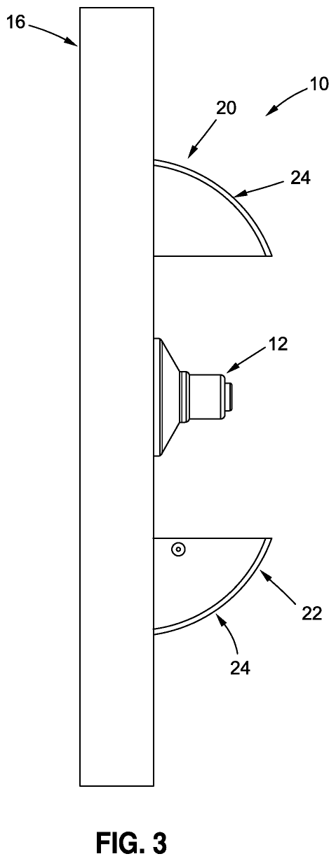

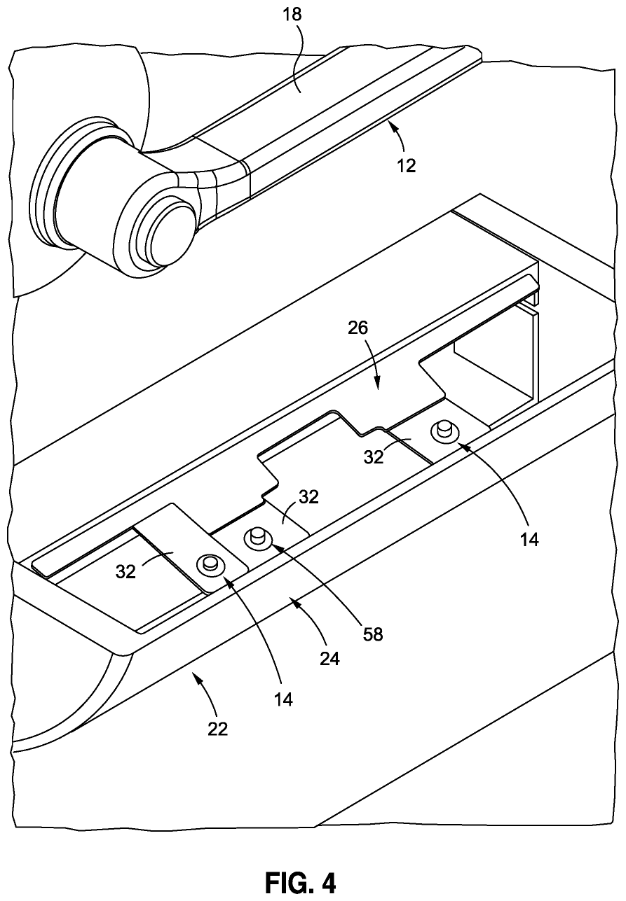

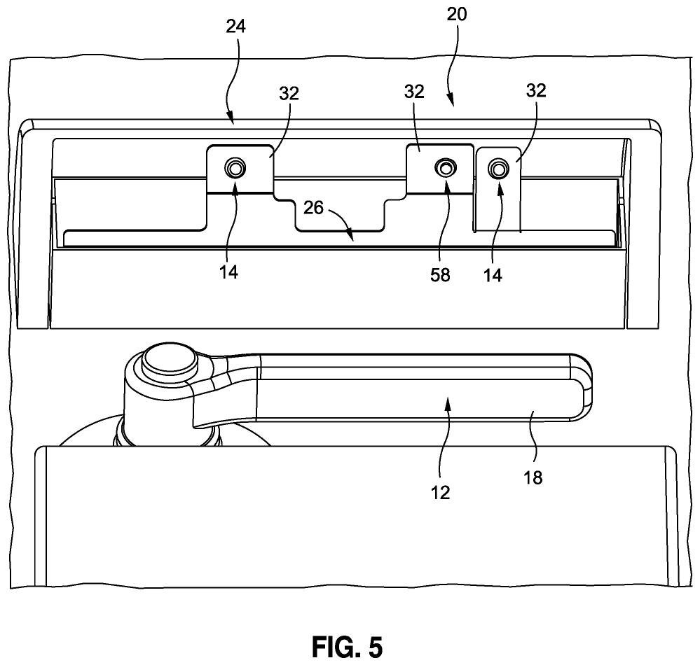

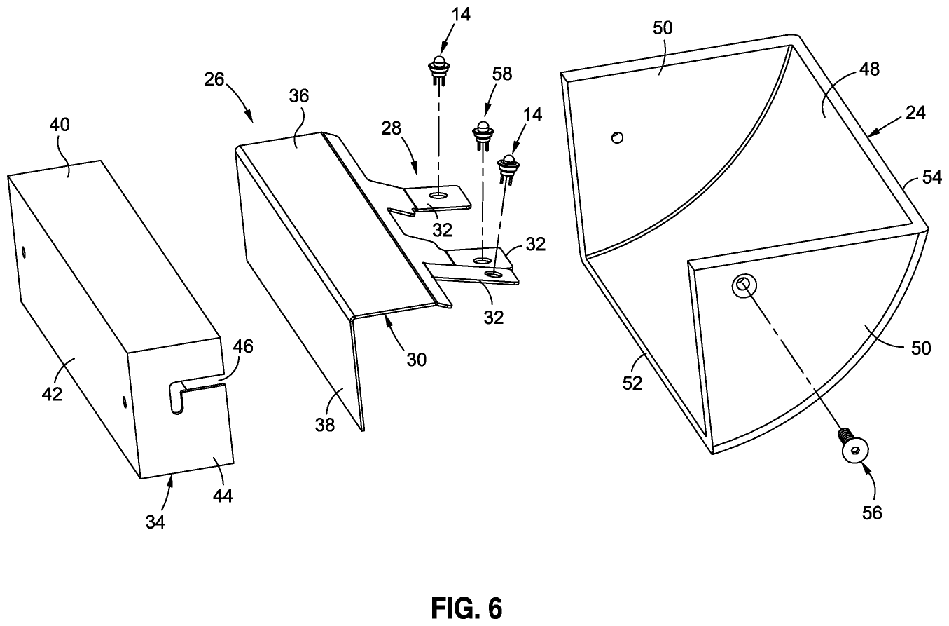

FIGS. 1 to 6 depict sanitizing apparatus 10 configured to emit UV light having a wavelength of 222 nanometers for sterilizing door handle 12. One or more sanitizing light elements 14 are mounted in close proximity to handle 12 for emitting such UV light to kill bacteria, viruses and other contaminants on handle 12. Apparatus 10 may be configured to detect the presence of a user's hand touching handle 12, and to automatically initiate a sanitizing cycle after the user moves his hand/fingers away from handle 12.

Apparatus 10 is configured for use with door 16 having a user-actuated opening mechanism 12, including a gripping surface/target surface 18 which interfaces with a user for opening door 16. In this text, the term “opening mechanism” may refer to a door handle, a faucet handle, an elevator button, a biometric security device, a keypad/keyboard, and other handles, buttons or other grippable/engageable elements. In FIGS. 1 to 6, opening mechanism 12 includes a rotatable handle having an elongate gripping portion defining a target/gripping surface 18 for a user to actuate handle 12.

Apparatus 10 includes a first, upper sanitizing assembly 20 mounted on door 16, and positioned above handle 12, and a second, lower sanitizing assembly 22 mounted on door 16, and positioned below handle 12. Each sanitizing assembly includes shield 24, chassis 26, and at least one sanitizing UV light element 14 for emitting light at 222 nanometers. Sanitizing assemblies 20, 22 are coupled to the door 16 close to handle 12 such that sanitizing light elements 14 are in sanitizing range of handle 12.

Elements 14 may be positioned about one to five inches away (i.e., above or below) from handle 12, leaving adequate space for a user to grasp and actuate handle 12. In this embodiment, a lower sanitizing assembly 22 extends outwardly from door 16 a greater distance than upper sanitizing assembly 20. This configuration allows the sanitizing light assembly 20 to produce a larger area of sanitizing light than the sanitizing light element 14 in upper sanitizing light assembly 20.

However, in other embodiments, sanitizing light elements 14 may be positioned closer, or farther away from handle 12, and still effectively sanitize handle 12.

Sanitizing light elements 14 are coupled to a chassis 26, which includes a mounting portion 28 and a bracket portion 30. Mounting portion 28 includes one or more mounting tabs 32 for engaging with individual sanitizing light elements 14. Each mounting tab 32 includes an aperture configured to receive a sanitizing light element 14. The tabs 32 extend outwardly from bracket portion 30 to position the sanitizing light elements 14 in an optimal position for sanitizing handle 12.

To that end, the sanitizing light elements 14 may be positioned a distance one to five inches away from handle 12, whether above or below, and may also be selectively oriented a distance extending perpendicular from door 16 such that light element 14 of the upper sanitizing assembly 20 extends approximately one to three inches perpendicular from door 16, and lower sanitizing assembly 22 extends a distance of at least three inches perpendicular from door 16.

Bracket portion 30 is configured for connecting chassis 26 to shield 24. Bracket portion 30 is configured to be placed within housing 34 which is connectable to door 16. Here, bracket portion 30 includes upper bracket wall 36 and rear bracket wall 38, Wall 38 is complementary to upper housing wall 40 and to rear housing wall 42. With bracket portion 30 placed within housing 34, upper bracket wall 36 is positioned adjacent upper housing wall 40, and rear bracket wall 38 is positioned adjacent rear housing wall 42.

Housing 34 includes two sidewalls 44 disposed on opposed ends of housing 34. Each sidewall 44 includes a slot 46 formed therein for mounting housing 34 within shield 24.

Shield 24 is positioned beside sanitizing light elements 14 to reflect light toward handle 12. Shield 24 includes arcuate wall 48 disposed between two opposed end walls 50. Wall 48 extends approximately 90 degrees and includes a lower edge 52 which may be positioned adjacent door 16. Wall 48 also includes upper edge 54 which extends away from door 16 to define an opening therebetween through which sanitizing light may be emitted.

Each shield end wall 50 includes an aperture formed therein which is configured to receive a mechanical fastener 56, such as a screw, nail or rivet. Fastener 56 is advanced through the respective shield end wall 50 and is configured to slide within slot 46 formed within housing sidewall 44 to connect housing 34 to shield 24.

Apparatus 10 includes motion sensor 58 configured to detect placement/removal of a user's hand or fingers near handle 12. Motion sensor 58 may be mounted to chassis 26 on mounting tab 32 to position sensor 58 near handle 12. Sensor 58 may employ such detection means as infrared and sonar, for detecting placement/removal of a user's hand near handle 12. Upon detection of a user's hand, motion sensor 58 may generate a first detection signal, and, upon detection of removal of a user's hand, sensor 58 generates a second detection signal. The first and second detection signals may be used to control operation of the sanitizing light elements 14.

Controller 60 is in operative communication with motion sensor 58 and sanitizing light element(s) 14 for controlling operation of sanitizer(s) 14 based on information received from the motion sensor 58. Controller 60 includes the memory capacity and processing power to generate a sanitizing cycle command in response to detection, by the motion sensor, of placement and removal of the hand or fingers of a user near handle 12.

Sanitizing light elements 14 are in communication with controller 60 for receiving a sanitation cycle command, and are adapted to emit UV light toward the handle 12 for sanitizing handle 12 in response to receipt of a sanitizing cycle command.

Controller 60 may be configured to generate a stop command in response to detection of placement of a user's hand near handle 12 during emission of light by first sanitizing light element 14. The first sanitizing light element 14 may be configured to stop emitting light in response to receipt of the stop command.

Apparatus 10 may include a local power source 62, such as, a battery, for providing power to controller 60, motion sensor 58, and sanitizing light elements 14. Alternatively, apparatus 10 may receive power from an outside power source, such as a power outlet or through a hard wire connection to a building's power source.

Apparatus 10 may be retrofitted onto existing surface, such as the surfaces of doors and banks of elevator buttons, or may be incorporated into newly-built doors or banks of elevator buttons.

Operation of apparatus 10 is activated by placement of a user's hand/fingers within the field of emitted light from the apparatus. This field is defined by motion sensor 58, which detects a user's hand as the user reaches for and grasps handle 12. When the motion detector 58 detects the placement of a user's hand near handle 12, motion sensor 58 generates the first detection signal, which is then communicated to controller 60. At this point, controller 60 may be placed on standby to wait for removal of the user's hand. Once motion sensor 58 detects removal of the user's hand from the detectable field, a second detection signal is generated and communicated to controller 60.

Upon receipt of the second detection signal, controller 60 initiates a sanitizing cycle. This cycle continues for a prescribed period of time of irradiation of handle 12. Controller 60 emits a sanitizing cycle command to light elements 60, which causes light elements 14 to emit light at 222 nanometers for the prescribed period of time. Thereafter, After passage of the prescribed period of time, elements 60 cease emitting light, and the cycle is complete.

During a sanitizing cycle, motion sensor 58 continues to detect placement of a user's hand near handle 12. If motion sensor 58 detects a user's hand during such a cycle, motion sensor 48 generates the first detection signal which is communicated to controller 60. If controller 60 receives a first detection signal during a sanitizing cycle, controller 60 generates and transmits a stop signal to light elements 14 to stop emitting light. Once motion sensor 58 detects removal of a user's hand from handle 12, and away from the detectable field, controller 60 initiates a sanitizing signal.

When a sanitizing cycle ends, apparatus 10 remains in standby mode, ready to be activated upon detection of a user's hand/fingers by the motion sensor. Apparatus 10 may sanitize the surface of up to 99% of germicidal properties.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

Where a range of values is provided, every intervening value, between the upper and lower limit of that range and any other stated or intervening value in that stated range is included within the invention. The upper and lower limits of these smaller ranges may be included in the smaller ranges, and are likewise included within the invention. Where the stated range includes one or both of the limits, ranges excluding either or both of those included limits are also included in the invention

In this specification and in the appended claims, the singular forms “a”, “and”, “said”, and “the” include plural referents unless the context clearly dictates otherwise. The claims may be so-drafted to require singular elements or exclude any optional element. These statements provide antecedent basis for use of such exclusive terminology as “solely”, “only”, and the like in connection with the recitation of claim elements, and/or the use of “negative” claim limitation(s).

While this invention has been described in conjunction with the exemplary embodiments outlined above, the foregoing description of exemplary embodiments of the invention, as set forth above, is illustrative, not limiting. The invention is not necessarily so constrained. Many alternatives, adaptations modifications, and/or variations may be apparent to those skilled in the art.

1. A sanitizing apparatus configured, upon activation, to emit UV light at a wavelength in the range of about 200 to about 225 nanometers from a suitable UV light source in the apparatus, and to direct said UV light onto a surface that repeatedly/regularly/often comes into contact with human hands/fingers/body parts.

2. The sanitizing apparatus of claim 1 wherein said wavelength is 222 nanometers.

3. The sanitizing apparatus of claim 1 further comprising a motion sensor for detecting the presence of said human hands/fingers/body parts near said light source.

4. The sanitizing apparatus of claim 1 further comprising at least one additional UV light source that emits UV light having a wavelength of at least 250 nanometers upon activation of said additional UV light source.
